(19) United States
(12) Patent Application Publication
(10) Pub. No.: US 2017/0105833 A1
(43) Pub. Date: Apr. 20, 2017

(54) A ureteral stent

(71) Applicant: Coloplast A/S, Humlebaek (DK)

(72) Inventors: Sebastien Seguy, Gourdon (FR); Thomas Beilvert, Saint Martial de Nabirat (FR); Jacques Millet, Sarlet la Canéda (FR); Chaabane Bougherara, Frederiksberg C (DK); Olivier Chouarche, Vitrac (FR)

(21) Appl. No.: 15/302,015

(22) PCT Filed: Apr. 10, 2015
(86) PCT No.: PCT/DK2015/050087

§ 371 Date: Oct. 5, 2016

(30) Foreign Application Priority Data
Apr. 11, 2014 (EP) 14290105.7
Jul. 23, 2014 (EP) 14178201.1

(51) Int. Cl. A61F 2/04; A61M 27/00; A61F 2/94

(52) U.S. Cl. CPC A61F 2/04; A61F 2/94; A61M 27/008; A61F 2250/0036; A61F 2002/048

(57) ABSTRACT

Disclosed is a ureteral stent having a body and a tail. The body of the stent has a renal section intended to be placed in a kidney of a patient, a ureteral section intended to be placed in at least part of a ureter of the patient, and a proximal section arranged at a proximal end of the body of the stent. The tail has at least one thread intended to end in the bladder of the patient. The proximal section (16) has a flexibility greater than the flexibility of the ureteral area of the stent.

### BACKGROUND

The present invention relates to a ureteral stent intended to maintain the flow of urine between the kidneys and the bladder of a patient.

In a person, urine secreted by the kidneys passes through the ureters to the bladder and is then evacuated from the body through the urethra during micturition. In a healthy person, the urine is evacuated from the kidney to the bladder in one direction, by means of peristaltic movements of the ureter.

Certain urological disorders or certain diseases may prevent this evacuation in the direction of the bladder. These disorders may in particular be due to the presence of a calculus or tumour or an obstruction of the pyeloureteral junction. In this case, the flow of urine to the bladder may be difficult or may no longer be possible at all. The urine remains in the kidney, which dilates and may cause nephritic colic. To remedy this disorder, a stent can be placed in the ureter in order to re-establish the function of the latter and to permit evacuation of the urine.

Patients and surgeons would welcome improvements relating to stents.

### BRIEF SUMMARY

The application relates to a ureteral stent according to claim 1.

### BRIEF DESCRIPTION OF THE DRAWINGS

The present invention and its advantages will be better understood with reference to the detailed description and with reference to the attached figures, in which:

FIG. 1 is a schematic view of a stent according to one embodiment when fitted in place in a patient;

FIG. 2 is an overall view of a ureteral stent according to one embodiment;

FIG. 3 is a sectional view of a part of the stent according to one embodiment;

FIG. 4 is a profile view of a stent according to one embodiment;

FIG. 5 is an enlarged view of a detail of the proximal section of the embodiment of FIG. 4;

FIG. 6 is a top view of one embodiment of the stent;

FIG. 7 is a sectional view of a part of the embodiment of FIG. 6;

FIG. 8 shows a stent according to one embodiment;

FIG. 9 is a sectional view of a part of the proximal section of the embodiment of FIG. 8;

FIG. 10 is a perspective view of the part of the embodiment of FIG. 9;

FIG. 11 is a perspective view of a stent according to one embodiment;

FIG. 12 shows a front view of the embodiment of FIG. 11;

FIG. 13 is a perspective view of a detail of the proximal section of the embodiment of FIG. 11;

FIG. 14 is a profile view of a stent according to one embodiment;

FIG. 15 is an enlarged view of part of the proximal section of the embodiment of FIG. 14;

FIG. 16 is an enlarged view of one embodiment of the proximal section of the stent;

FIG. 17A is a side view of one embodiment of the proximal section of the stent;

FIG. 17B is a sectional view of the embodiment of FIG. 17A;

FIG. 18A is a side view of one embodiment of the proximal section of the stent;

FIG. 18B is a sectional view of the embodiment of FIG. 18A;

FIG. 19A is a schematic side view of one embodiment of the proximal section of the stent;

FIG. 19B is a schematic side view of the embodiment of FIG. 19A rotated 90 degrees; and

FIG. 19C is an end view of the proximal section of the stent according to FIGS. 19A and 19B.

### DETAILED DESCRIPTION

The ureteral stent of this application includes a body and a tail, the body having a renal section for placement in a kidney of a patient, a ureteral section for placement in at least part of a ureter of said patient, and a proximal section arranged at a proximal end of the body of the stent, the tail having at least one thread configured to end in the bladder of said patient.

The embodiments relate to a stent which preserve the peristaltic movements of the ureter, thereby preventing reflux of urine in the direction of the kidneys. The stent avoids irritation of the bladder and irritation caused by friction in the ureter. The stent is therefore better tolerated by patients.

The ureteral stent as includes a tubular proximal section of cylindrical shape and annular cross section, and has a flexibility greater than the flexibility of the ureteral section of the stent.

The ureteral stent is useful for placement in a ureter of a patient suffering from a urological disorder or disease, such as a calculus, a tumour, or an obstruction of the pyeloureteral junction in particular. The stent extends as far as the kidney and has a curved renal section, the curved shape allowing the stent to be more effectively held in place in the kidney. The stent has a ureteral section inserted in the ureter of the patient. The ureteral section extends beyond the location of the urological disorder, and ensures a flow path in the defective part of the ureter. One end of the stent is provided with a tail which comprises at least one thread configured to end in or near the bladder. The thread is suitable for permitting evacuation of the urine and has a sufficiently fine diameter to be virtually physically imperceptible to the patient.

In the following disclosure, the features of the various exemplary embodiments are suitable to be combined with each other unless specifically noted otherwise.

In the following disclosure, end means an end-most location and end portion means that segment adjacent to and near the end of an object.

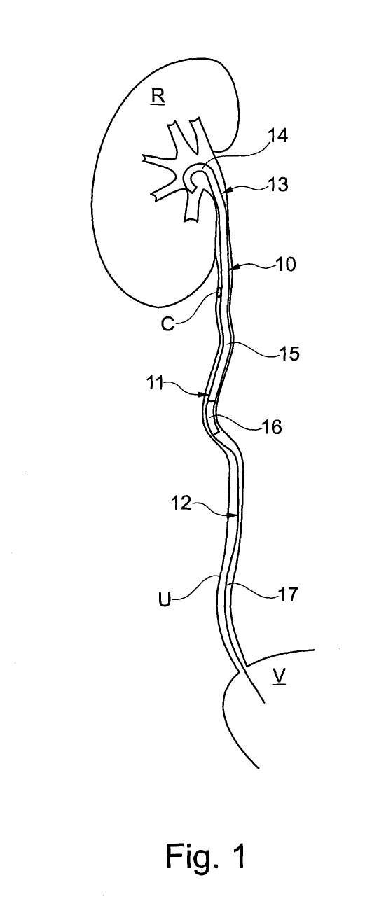

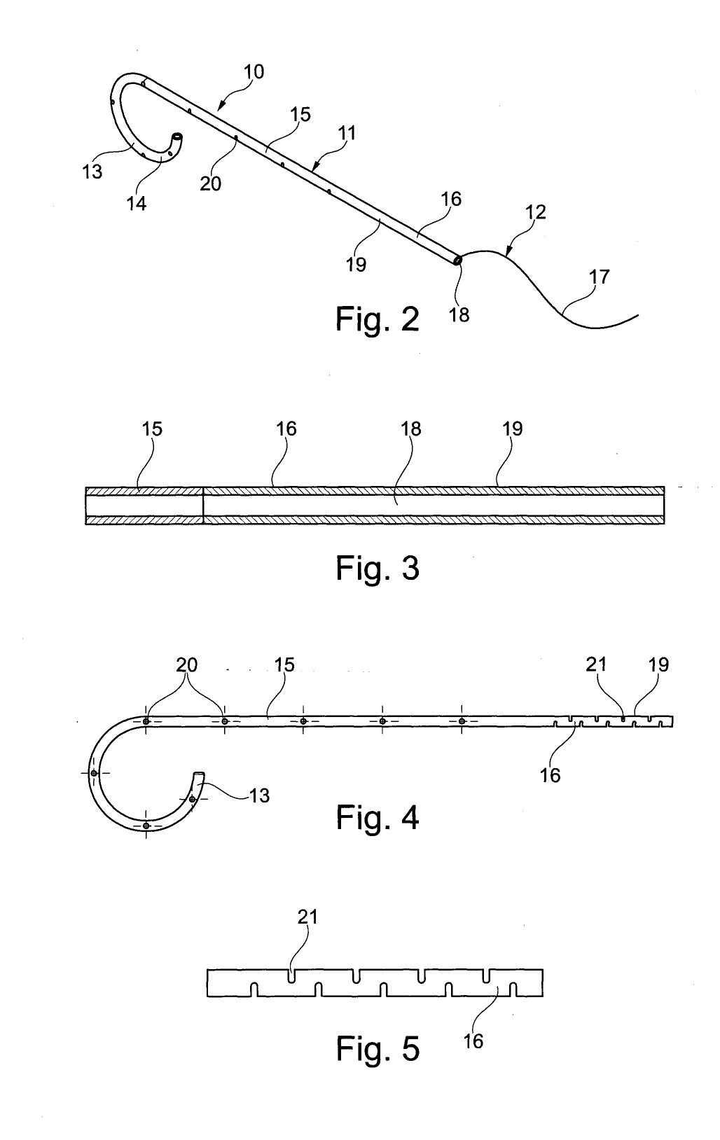

FIG. 1 is a schematic view of one embodiment of a ureteral stent 10, placed in a patient presenting a calculus C. FIG. 2 illustrates a similar stent before it is fitted in place in a patient.

With reference in particular to FIGS. 1 and 2, the ureteral stent 10 has a body 11 and a tail 12. The body 11 has a renal section 13 for placement in a kidney of a patient. The renal section 13 has a curved end portion 14 and improves retention of the stent. This part of the stent is physically imperceptible or virtually imperceptible to the patient and, as a result, does not cause any discomfort or pain.

In one embodiment, the stent includes a ureteral section 15 for placement in a ureter U of the patient. Embodiments include stents manufactured with varying lengths of at least the ureteral section such as to accommodate variations in patient physiology. As has been indicated above, a ureteral stent of this kind is suitable for placement in a patient in cases of disease or obstruction in the area of the ureter. The obstruction may be due to the presence of a calculus C, as is illustrated by FIG. 1, a tumour or a constriction in particular. The length of the ureteral section 15 of the stent should be sufficient to ensure that, after placement of the stent, the body 11 thereof extends beyond the site of the calculus C or the tumour in the direction of the bladder V.

In one embodiment, the body 11 of the stent additionally has a proximal section 16 which is integral with the ureteral section 15, at the end away from the renal section 13. The proximal section 16 is situated in continuation of the ureteral section 15.

In one embodiment, the tail 12 of the stent 10 is formed by at least one thread 17 or suture configured to extend from the proximal section 16 in the direction of the bladder V, when the stent is placed in a patient.

In embodiments, the thread permits flow of urine from the kidney R to the bladder V without permitting flow in the opposite direction from the bladder to the kidney. This avoids the aforementioned problems, particularly the sensation of having to pass urine. The thread 17 causes a dilation of the ureter and, as a result, permits easier and therefore less painful evacuation of the calculi.

In one embodiment, the tail 12 includes a single thread, as illustrated in FIG. 1. In one embodiment, it includes two or more threads. The tail 12 is configured to end in the bladder and extend a few centimetres into the bladder when the stent is placed in a patient. In one embodiment, the tail extends 5 to 6 centimetres into the bladder.

In embodiments wherein the tail is formed by two or more threads, the threads are free and independent of one another. In other embodiments, the threads are fixed to one another, for example by a knot. The knot is suitably located near the proximal section 16, in which case it will be positioned in the ureter during the use of the stent. Alternatively, the knot is located near the ends of the threads extending away from the proximal section, in which case it will be positioned in the bladder during the use of the stent. Other ways of connecting the threads to one another are also acceptable, for example, but not limited to, braiding.

In one embodiment, the ureteral section 15 and the proximal section 16 are tubular and cylindrical, that is to say they have a substantially annular cross section, with a longitudinal channel 18 defined by a lateral wall 19. In one embodiment, the lateral wall has, at least in some areas, through-openings 20 which allow the urine to flow from outside the stent to the inside of the longitudinal channel, and vice versa. In one embodiment, the longitudinal channel 18 opens out at the end of the proximal section 16, near the tail 12. In one embodiment, the renal section 13 also has a channel, and openings passing through the lateral wall 19. The channel 18 serves, during the placement of the stent, for introduction of a guide and, during the use of the stent, for evacuation of urine.

In one embodiment, and with reference to FIGS. 1 and 3, the proximal section 16 of the stent is flexible. In particular, this proximal section is more flexible than the ureteral section 15.

In this application, flexibility is defined as the resistance to elastic deformation of a body. The more a body is flexible, the less the force that has to be applied to it to obtain a given deflection. In the present specification, stiffness or rigidity is considered the inverse of flexibility.

The flexibility can be measured in the following way: the specimen for which flexibility is being determined is placed on two punctiform supports that are separated by a distance L. A downward force P is applied in the centre of the specimen. The deformation U_{y} of the specimen is measured by measuring the movement of the centre of the specimen under the effect of the force P. This deformation, as a function of the force, allows a curve to be established. The coefficient of flexibility is defined as being the slope of the tangent to this curve, at the origin.

From the mathematical point of view, the coefficient of flexibility is defined by

$$K = \frac{{L}^{3}}{48} \cdot \frac{P}{\vert {U}_{y} \vert}$$

In one embodiment, the coefficient of flexibility of the proximal section of the stent is less than or equal to 200 N mm².

The more flexible the stent, the greater the flexibility and the lower the coefficient of flexibility.

Flexibility of the proximal section is suitably obtained by use of flexible materials and/or by providing the proximal section with a shape that gives it flexibility.

In one embodiment, the proximal section of the stent is configured to allow the body 11 of the stent to match the shape of a ureter U, in particular in non-rectilinear parts of the ureter, during movements of the patient, especially movements caused by respiration. The stent and the ureter are configured to allow relative shifting between them. For this purpose, the proximal section 16 of the stent is sufficiently flexible to be able to follow the ureter.

In one embodiment, the ureteral section 15 of the stent has a flexibility allowing it to adapt to the sinuosities of the ureter. Materials suitable for producing the stent include polymers such as polyurethane, copolymers such as polyether block amide known by the name PEBA, polyamides, silicone, polyolefins sold under the names INFUSE™, VISTAMAXX™, QUEO™ or NOTIO™, polyamides, Poly Vinyl Chloride (PVC,) thermoplastic polyurethanes, aromatic polyethers, aromatic and aliphatic polyesters having a Shore hardness of generally between 25 and 95, compounds based on thermoplastic elastomers, vulcanized thermoplastic elastomers, mixtures and alloys based on thermoplastic polyurethane, polymers and copolymers sold under the names THERMOFLEX™, HYTRIL™, ARNITEL™, Ethylene Vinyl Acetate (EVA), and thermoplastic elastomers known by the acronyms SIS, SEBS, SEPS, SEEPS, SBS, SIBS or SIBSTAR.

In embodiments, an external diameter of the ureteral stents is between 1.5 mm and 4 mm.

In one embodiment, illustrated in FIGS. 1 and 3, the proximal section 16 is made of a flexible material. This material can be chosen from among different types of polymers such as polyurethane, copolymers such as polyether block amide (PEBA), polyvinyl chloride, polyamides or silicone in particular, or more generally from among the materials mentioned above for the ureteral section.

Suitable materials for the thread (or suture) include: polyethylene, polyamide, polyester, silk, steel, resorbable material (such as polyglactin acid), high-density polypropylene, meta-aramid and para-aramid, such as KEVLAR™ or NOMEX™.

In embodiments, the thread is configured to have a diameter ranging between 0.16 mm and 1.3 mm. In one embodiment, the diameter substantially equal to 0.2 mm.

When the stent 10 is implanted in a patient, the thread 17 serves to help the flow of urine from the kidney to the bladder, without permitting flow in the opposite direction. The thread also facilitates the evacuation of calculi, since it causes a dilation of the ureter. Another function of the thread is to permit the withdrawal of the stent when the stent is to be removed from the patient. In one embodiment, the thread 17 is sufficiently solid to allow the stent to be withdrawn by pulling on the thread. It is also possible to use two or more threads in order to for withdrawal of the stent.

In embodiments, the threads are fixed to the body 11 of the stent at different locations, particularly in the ureteral section 15 or in the proximal section 16 of the stent. One advantage of fixing the thread to the proximal section 16 is that the thread is always in contact with an edge of the proximal section 16. This provides for an easy flow of urine. Moreover, this proximal section 16 is easily accessible in order to fix the thread to it.

Another advantage is the fact that a pull applied to the tail end of the proximal section eliminates the possibility of the latter folding or rolling up during the withdrawal of the stent.

In embodiments, the thread is fixed to the end of the ureteral section 15. This section is more rigid than the proximal section and it is therefore advantageously possible to apply a stronger pull to the thread.

In one embodiment, the thread can be fixed to the ureteral section 15 and to the proximal section 16. This makes it possible to fix the thread to the body of the stent, while at the same time providing for the thread to remain near the edge of the proximal section and avoid rolling up of the stent during withdrawal.

The fixing of the thread to the stent can be obtained in different ways. In one embodiment, the thread is passed through the wall of the body of the stent and knotted either to the wall or to another part of the thread. In one embodiment, the thread is adhesively bonded to the body of the stent. In one embodiment, the thread is fixed to the body by welding.

The flexibility of the material forming the proximal section 16 of the stent can be obtained in several different ways. In one embodiment, the proximal section 16 is made of a material different from that forming the rest of the body 11 of the stent 10. In one embodiment, the proximal section is made more flexible by subjecting it to a separate operation, for example a chemical action. In one embodiment, the proximal section of the stent is made of polyurethane, and the separate operation on the proximal section includes immersion in a plasticizer of the cyclohexanone type, the effect of which is to soften the material and increase the flexibility.

Alternatively, the stent can be produced with a body 11 made of a flexible material wherein the whole body of the stent except for the proximal section 16 is subjected to a hardening process. The hardening can be obtained by the action of a chemical component, by exposure to light, for example UV light, or by exposure to heating or cooling.

FIGS. 4 and 5 illustrate another embodiment of a stent 10 having a flexible proximal section 16. In contrast to the embodiment illustrated in FIGS. 1 and 3, this flexibility is not provided by the material used or by a separate process, but instead by the shape of the proximal section 16. In one embodiment, the proximal section has openings 21 passing through the lateral wall 19. In one embodiment, the openings are transverse slits, that is to say slits arranged in a plane perpendicular to a longitudinal axis of the proximal area 16. The presence of these openings has the effect of allowing the proximal area to bend, which allows it to easily follow the sinuosities of the ureter, in particular when relative movements of the ureter and of the stent occur.

In one embodiment, the flexibility of the proximal section 16 is provided as a combination of its shape and by the choice of material. In one embodiment, transverse openings 21 are formed in a proximal section 16 that is made of a flexible material.

In embodiments, the thread 17 or suture is configured to be fixed to the proximal section 16 or to the ureteral section 15. The advantages of this fixing are the same as those that have been described with reference to the embodiment in FIGS. 1 and 3.

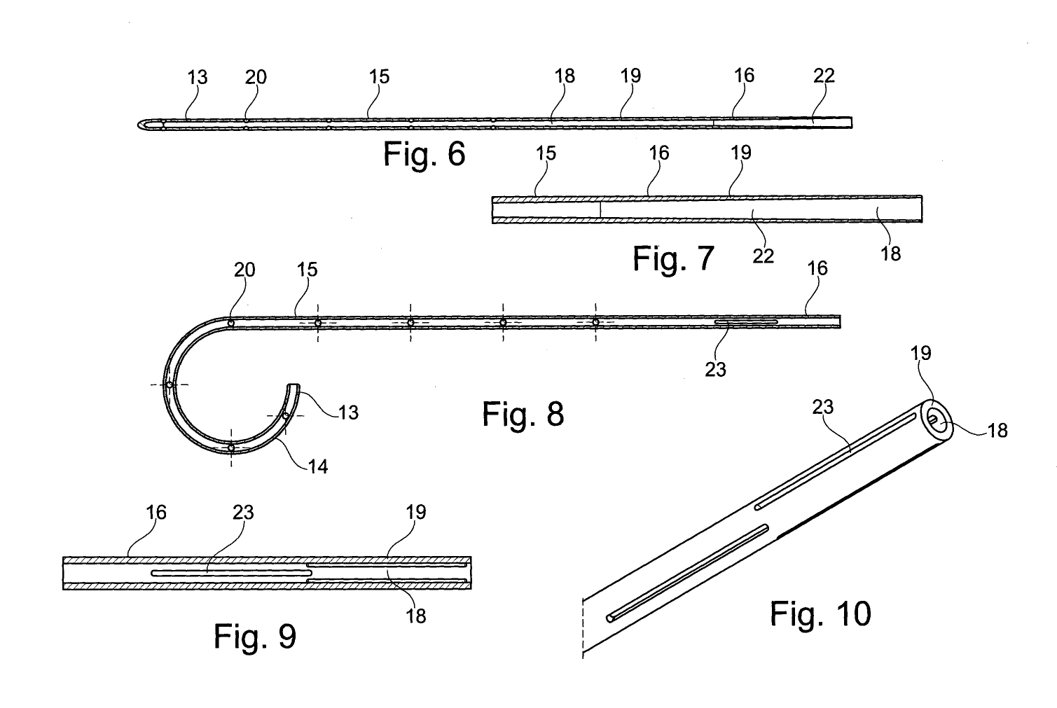

In the embodiment illustrated in FIGS. 6 and 7, the proximal section 16 is cylindrical and includes a conical inner recess 22. This recess has an axis coincident with the longitudinal axis of the cylinder. The thickness of the lateral wall 19 forming the proximal section 16 decreases in the direction towards the tail of the stent. In this way, the end of the proximal section opposite the ureteral section has a very small thickness and, therefore, high flexibility. Thus, the proximal section can easily follow the movements of the ureter.

FIGS. 8, 9 and 10 show one embodiment in which the proximal area 16 has through-openings made in the form of longitudinal slits 23 passing through the lateral wall 19. These longitudinal slits 23 allow the proximal area 16 to deform easily, so as to follow the shape of the ureter. In one embodiment, the slits 23 do not extend all the way to the end of the proximal area 16, meaning that the end forms a complete ring.

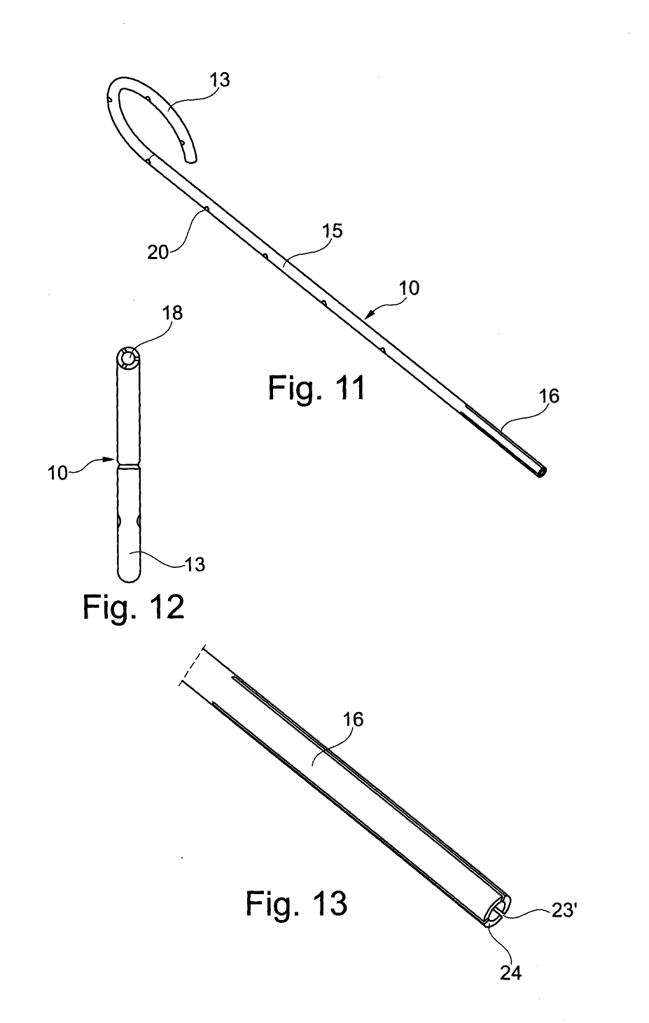

In one embodiment illustrated in FIGS. 11, 12 and 13, the proximal section includes through-openings made in the form of longitudinal slits 23′. However, at least some of these slits extend all the way to the tail end of the stent in such a way as to form tongues 24 in the proximal section. The tail end therefore has a slit annular shape. The proximal section 16 is flexible and able to adapt to the shape of the ureter.

In one embodiment, a thread is attached to the proximal section, more precisely to one of the tongues 24. In one embodiment, more than one thread is attached in such a way that all the tongues of this proximal section are connected to the threads forming the tail of the stent. This has the advantage of allowing the tongues to float in the ureter when the stent is in place in the patient in use, while also drawing these tongues together when the tail of the stent is pulled to extract it from the patient.

In one embodiment illustrated in FIGS. 17A-17B and 18A-18B, the proximal section 16 of the stent includes one or more openings or slits 29 and forming one or more tongues 28. In embodiments, two or more openings or slits 29 are distributed such that two or more tongues 28 describe equally large arcuate portions. In one embodiment, the proximal section 16 includes a single (one and only one) tongue 28 providing flexibility.

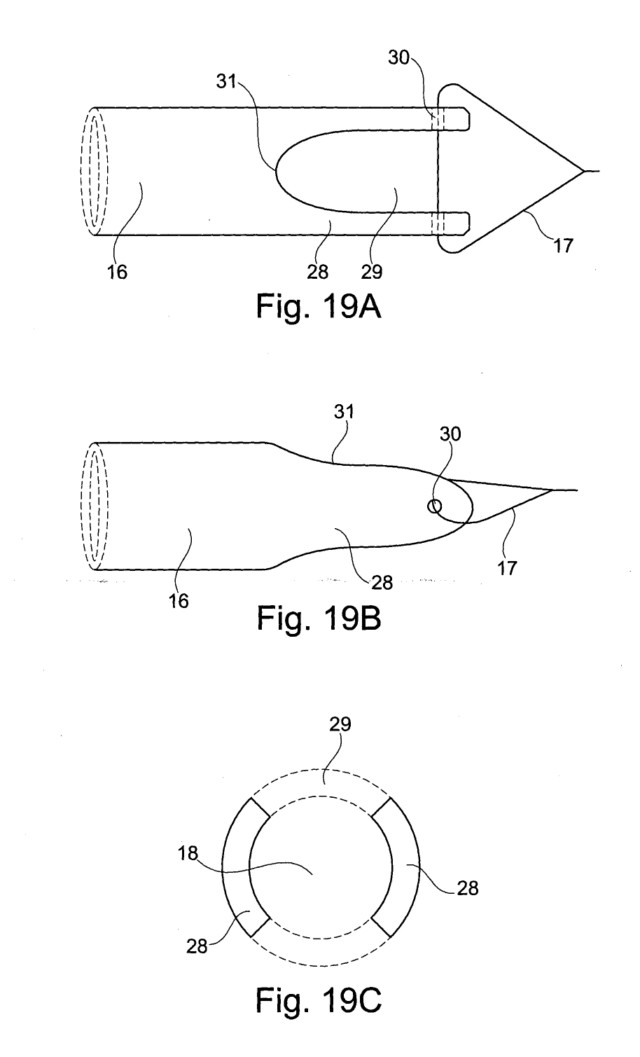

In one embodiment illustrated in FIGS. 19A-19C, the proximal section 16 of the stent includes two tongues 28 separated by openings or slits 29. In one embodiment, the proximal section 16 is provided with openings 29 that has the proximal section 16 reflect the shape of an “inca hat”/“Phrygian hat”.

In one embodiment, a contour 31 of the one or more tongues 28 is rounded and may additionally be rounded toward the tail end of the proximal section 16 thereby providing a smooth shape with a flexibility that is able to adapt to the shape of the ureter in turn assisting in removing patient discomfort. In one embodiment, a thread 17 is attached to the tongues 28 through one or more threading passages 30. FIG. 19C also illustrates the longitudinal channel 18 of the stent.

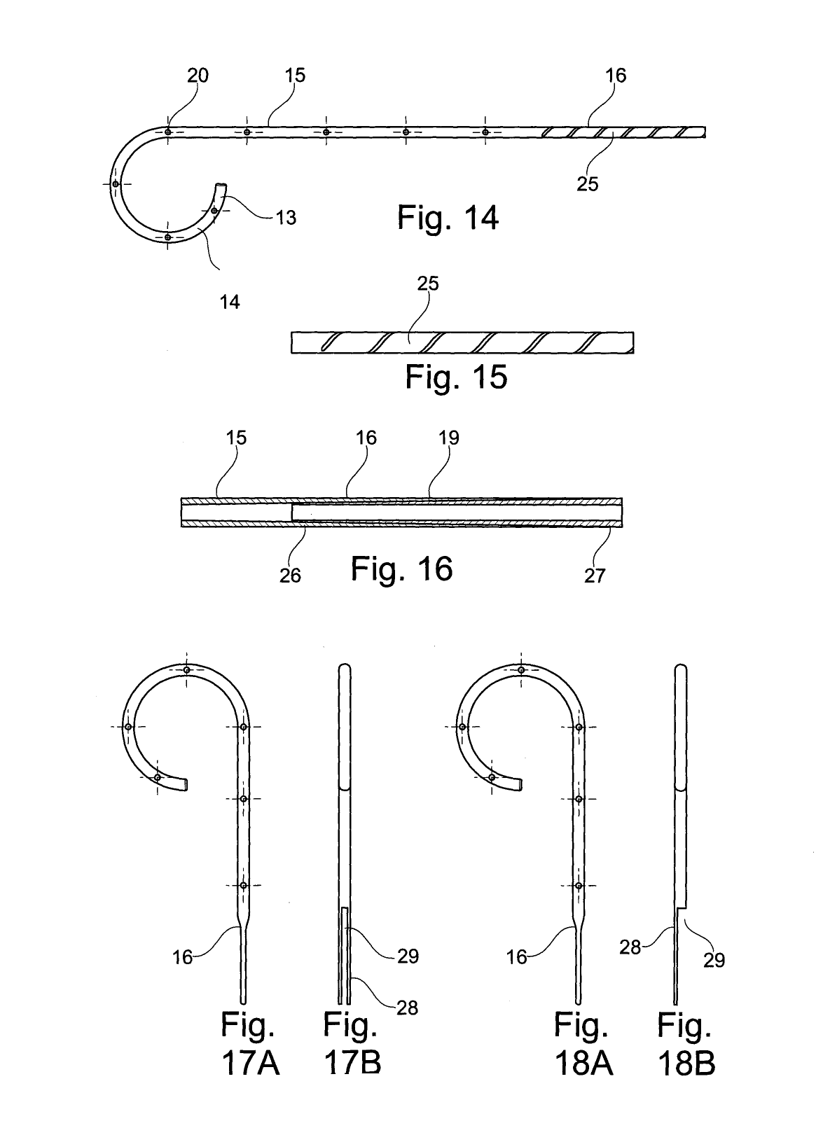

In one embodiment illustrated in FIGS. 14 and 15, the proximal section 16 of the stent is formed by a spiral 25. This spiral shape allows flexing of the proximal section and thus provides high flexibility. By virtue of the spiral shape, the stent can follow the sinuosities of the ureter of the patient. In one embodiment, the tail 12 of the stent is attached to one of the turns of the spiral of the proximal section 16. The turns of the spiral of the proximal area can be unwound to make withdrawal of the stent easier. In one embodiment including the spiral shape, the thread is fixed to the ureteral section 15 of the stent.

If unwinding of the spiral during withdrawal of the stent is not desired, in one embodiment the thread is fixed to several or all of the turns of the spiral 25 forming the proximal section. This makes it possible to retain the flexibility while at the same time avoiding too much deformation of the stent.

In one embodiment, two threads are provided, one thread being fixed to the proximal section 16 and the other to the ureteral section 15. The thread fixed to the proximal section is able to guide the urine during the use of the stent and to guide the proximal section during the withdrawal of the stent. The thread fixed to the ureteral section makes it possible to retract the body of the stent without deforming it and also makes it possible to limit the deformation of the proximal section during withdrawal.

In one embodiment illustrated in FIG. 16, the proximal section 16 is produced, for example, by co-extrusion of two materials having different degrees of flexibility. The thickness of the first material 26, having a defined coefficient of flexibility and being used to form the ureteral section 15, decreases in the direction of the tail end of the stent, while the thickness of the second material 27, having a coefficient of flexibility lower than that of the first material, increases in the direction of the tail end of the stent, in such a way that the total thickness of the two materials remains constant. In other words, less flexible material is progressively replaced by more flexible material in the direction of the free (tail) end of the stent.

A ureteral stent has been described which permits evacuation of urine from a patient while at the same time preventing reflux of the urine in the direction of flow towards the kidneys. The stent is configured to be placed to permit easier evacuation of calculi. The stent is virtually physically imperceptible to the patient due to the flexibility of the proximal section. The thread is fine and flexible, such that it is practically unnoticed by the patient.

### Embodiments

A. A ureteral stent comprising:
a body having a renal area configured to be placed in a kidney of a patient, a ureteral area connected to the renal area and configured to be placed in a ureter of the patient, and a proximal area connected to the ureteral area and located at a proximal end of the body; and
a tail comprising a thread connected to the proximal end of the body of the stent; wherein the proximal area is tubular and is provided with a first flexibility that is greater than a second flexibility of the ureteral area of the stent.
   B. The ureteral stent according to embodiment A, wherein the proximal area of the stent is comprises a material that is more flexible than a material forming the ureteral area of the stent.
   C. The ureteral stent according to embodiment B, wherein the proximal area of the stent is fabricated from two materials each having a different flexibility and the proximal area has a longitudinal channel of constant diameter defining a lateral wall of constant thickness.
   D. The ureteral stent according to embodiment C, wherein a first of the materials is less flexible than a second of the two materials, and a thickness of the first material decreases towards the tail of the stent and a thickness of the second material increases towards the tail of the stent.
   E. The ureteral stent according to embodiment A, wherein the proximal area comprises through-openings.
   F. The ureteral stent according to embodiment D, wherein the through-openings are slits.
   G. The ureteral stent according to embodiment F, wherein the slits extend in a longitudinal direction of the proximal area.
   H. The ureteral stent according to embodiment F, wherein the slits extend in a transverse direction of the proximal area.
   I. The ureteral stent according to embodiment E, wherein the through-openings are holes.
   J. The ureteral stent according to embodiment C, wherein the proximal area has a conical inner recess, such that a thickness of the lateral wall decreases towards the tail of the stent.
   K. The ureteral stent as claimed in embodiment A, wherein the proximal area is configured as a spiral shape.
   L. The ureteral stent according to embodiment C, wherein the proximal area comprises longitudinal recesses formed in the thickness of the lateral wall.
   M. The ureteral stent according to embodiment A, wherein the proximal area has a slit annular cross section.
   N. The ureteral stent according to embodiment A, wherein the tail is connected to the proximal area.
   O. The ureteral stent according to embodiment A, wherein the tail is connected to the ureteral area.
   P. The ureteral stent according to embodiment A, wherein a body portion of the stent in the proximal area has a coefficient of flexibility equal to or below 200 N mm².
   Q. Ureteral stent (10) having a body (11) and a tail (12), the body (11) having a renal area (13) intended to be placed in a kidney (R) of a patient, a ureteral area (15) intended to be placed in at least part of a ureter (U) of said patient, and a proximal area (16) arranged at a proximal end of the body (11) of the stent, the tail (12) having at least one thread (17) intended to end in the bladder (V) of said patient, characterized in that said proximal area (16) is tubular, has a cylindrical shape and is of annular cross section, and in that this proximal area (16) has a flexibility greater than the flexibility of the ureteral area (15) of the stent.
   R. Ureteral stent according to embodiment Q, characterized in that the proximal area (16) of the stent is made of at least one material more flexible than the material forming the ureteral area (15) of the stent.
   S. Ureteral stent according to embodiment R, characterized in that the proximal area (16) of the stent is made of two materials having different flexibility, in that this proximal area (16) has a longitudinal channel of constant diameter defining a lateral wall (19) of constant thickness, in that the material quantity of the less flexible material decreases towards the tail of the stent, while the material quantity of the more flexible material increases towards the tail of the stent.
   T. Ureteral stent according to any one of embodiments Q-S, characterized in that the proximal area (16) has a shape that gives it a flexibility greater than the flexibility of the ureteral area (15).
   U. Ureteral stent according to embodiment T, characterized in that the proximal area (16) has through-openings.
   V. Ureteral stent according to embodiment U, characterized in that said through-openings are slits (23, 23′).
   W. Ureteral stent according to embodiment V, characterized in that said slits (23, 23′) are longitudinal.
   X. Ureteral stent according to embodiment V, characterized in that said slits are transverse.
   Y. Ureteral stent according to embodiment U, characterized in that said through-openings are holes.
   Z. Ureteral stent according to embodiment S or T, characterized in that the proximal area (16) has a conical inner recess (22), such that the thickness of the lateral wall (19) decreases towards the tail of the stent.
   AA. Ureteral stent as claimed in embodiment Q, characterized in that the proximal area (16) is formed by a spiral (25).
   BB. Ureteral stent according to embodiment Q, characterized in that the proximal area (16) has longitudinal recesses formed in the thickness of the lateral wall (19).
   CC. Ureteral stent according to embodiment Q, characterized in that said proximal area (16) has a slit annular cross section.
   DD. Ureteral stent according to embodiment Q, characterized in that the tail (12) is rigidly connected to the proximal area (16).
   EE. Ureteral stent according to embodiment Q or DD, characterized in that the tail (12) is rigidly connected to the ureteral area (15).
   FF. Ureteral stent according to any one of the preceding embodiments Q-EE, characterized in that the proximal area (16) has a coefficient of flexibility of below 200 N mm2.
   GG. Method of producing a ureteral stent according to embodiment Q, characterized in that the proximal area (16) undergoes a treatment making it more flexible than the ureteral area (15).
   HH. Method of production according to embodiment GG, characterized in that the ureteral area (15) undergoes a treatment making it less flexible than the proximal area (16).
   II. Method of production according to embodiment GG or HH, characterized in that said treatment is a chemical treatment.
   JJ. Method of production according to embodiment GG or HH, characterized in that said treatment is a thermal treatment.

What is claimed is:

1. A ureteral stent comprising:
a body having a renal section configured to be placed in a kidney of a patient, a ureteral section connected to the renal section and configured to be placed in a ureter of the patient, and a proximal section connected to the ureteral section at a proximal end of the body; and
a tail comprising a thread connected to the body of the stent; wherein the proximal section is tubular and is provided with a first flexibility that is greater than a second flexibility of the ureteral section of the stent.

2. The ureteral stent according to claim 1, wherein the proximal section of the stent comprises a material that is more flexible than a material forming the ureteral section of the stent.

3. The ureteral stent according to claim 2, wherein the proximal section of the stent is fabricated from two materials of different flexibilities and the proximal section has a longitudinal channel of constant diameter defined by a lateral wall of constant thickness.

4. The ureteral stent according to claim 3, wherein a first of the two materials is less flexible than a second of the two materials, and a thickness of the first material decreases towards the tail of the stent and a thickness of the second material increases towards the tail of the stent.

5. The ureteral stent according to claim 1, wherein the proximal section comprises through-openings.

6. The ureteral stent according to claim 5, wherein the through-openings are slits.

7. The ureteral stent according to claim 6, wherein the slits extend in a longitudinal direction of the proximal section.

8. The ureteral stent according to claim 6, wherein the slits extend in a transverse direction of the proximal section.

9. The ureteral stent according to claim 5, wherein the through-openings are holes.

10. The ureteral stent according to claim 1, wherein the proximal section has a conical inner recess, such that a thickness of a lateral wall of the proximal section decreases towards the tail of the stent.

11. The ureteral stent as claimed in claim 1, wherein the proximal section is configured as a spiral shape.

12. The ureteral stent according to one of claim 3 or 10, wherein the proximal section comprises longitudinal recesses formed in the thickness of the lateral wall.

13. The ureteral stent according to claim 1, wherein the proximal section has a slit annular cross section.

14. The ureteral stent according to claim 1, wherein the tail is integral with the proximal section.

15. The ureteral stent according to claim 1, wherein the tail is integral with the ureteral section.

16. The ureteral stent according to claim 1, wherein a body portion of the stent in the proximal section has a coefficient of flexibility equal to or below 200 N mm².

17. The ureteral stent according to claim 1, wherein the tail is connected to the proximal end of the body.
